(19) United States
(12) Patent Application Publication
(10) Pub. No.: US 2015/0196591 A1
(43) Pub. Date: Jul. 16, 2015

(54) NUTRITIONAL FORMULATION FOR WEIGHT CONTROL

(71) Applicant: JJ ROBIN, LLC, Wilmington, DE (US)

(72) Inventor: Janet Zalman Kossow, Washington, DC (US)

(21) Appl. No.: 14/635,203

(22) Filed: Mar. 2, 2015

Related U.S. Application Data
(63) Continuation of application No. PCT/US2013/058884, filed on Sep. 10, 2013.
(60) Provisional application No. 61/699,366, filed on Sep. 11, 2012.

(51) Int. Cl. A61K 33/24; A61K 31/51; A61K 31/525; A61K 31/455; A61K 31/191; A61K 31/675; A61K 31/519; A61K 31/14; A61K 31/047; A61K 31/714; A61K 31/4188; A61K 31/196; A23L 1/302; A23L 1/305; A23L 1/304; A23L 1/29; A23L 1/30; A23L 1/308; A61K 31/198

(52) U.S. Cl. CPC A61K 33/24; A61K 31/198; A61K 31/51; A61K 31/525; A61K 31/455; A61K 31/191; A61K 31/675; A61K 31/519; A61K 31/14; A61K 31/047; A61K 31/714; A61K 31/4188; A61K 31/196; A23L 1/302; A23L 1/3051; A23L 1/304; A23L 1/293; A23L 1/3002; A23L 1/3081; A23V 2002/00

(57) ABSTRACT

A nutritional formulation or dietary regime for improving animal health. The nutritional formulation includes effective and dietary acceptable amounts of glucose tolerance factor (GTF) chromium and L-Glutamine that are beneficial in controlling animal weight and reducing blood sugar levels.

### RELATED APPLICATIONS

This application is a continuation of International application No. PCT/US2013/058884, filed Sep. 10, 2013, titled “NUTRITIONAL FORMULATION FOR WEIGHT CONTROL,” which claims priority from U.S. provisional application No. 61/699,366 filed Sep. 11, 2012, the entire contents of both of which are hereby fully incorporated herein by reference for all purposes.

### FIELD OF THE INVENTION

The present invention relates to the field of nutritional formulations and dietary supplements. More particularly, the present invention provides a nutritional formulation or dietary regime and method for making a nutritional formulation designed to promote weight control and control of blood sugar levels in animals.

### BACKGROUND

Diabetes and obesity now affect millions of Americans. One immediately apparent cause of these problems is overeating, often caused by food cravings, including persistent, uncontrolled cravings for sweets.

It is therefore desirable to provide something that can help people control and reduce cravings and lose weight. In particular it is desirable to provide a way to help people who have had persistent, uncontrolled cravings for sweets.

### SUMMARY

Nutritional formulations, dietary regimes and method for making nutritional formulations are disclosed. These nutritional formulations and dietary regimes are designed to assist animals, particularly humans, in losing weight and improving their overall health. More particularly, the nutritional formulations or dietary regimes include effective amounts of glucose tolerance factor (GTF) chromium and L-Glutamine that assist in promoting human weight control and the reduction of blood sugar levels.

When an individual's cravings decrease, by using the nutritional formulations/dietary regimes, they are able to stabilize their blood sugar, have more energy, decrease their appetite, and lose weight effectively.

These nutritional formulations and dietary regimes will reduce the current escalation of diabetes and obesity that now affect millions of Americans.

An objective of the present invention is to provide an improved nutritional and weight-loss supplement or aid and to provide a safe, easy, and effective method of weight loss that can lead to long lasting weight reduction.

Further objectives and advantages are to provide a nutritional supplement or dietary regime that can act to decrease an individual's cravings for sweets.

Still further objectives and advantages are to provide a nutritional supplement that can enhance weight loss and to provide various herbs, botanicals, minerals, coenzymes, and vitamins to improve health.

### DETAILED DESCRIPTION OF THE PRESENTLY PREFERRED EXEMPLARY EMBODIMENTS

### Definition and Usage of Terms

As used herein, the term “weight control” is intended to refer to the conveyance of one or more of the following benefits to an animal, in particular a mammal, specifically a human. These benefits include but are not limited to weight loss, body sculpting, reduction in cholesterol levels, reduction in blood sugar levels, reduction in blood glucose levels, skin firming benefits, anti-cellulite benefits, reduction in blood pressure, increase in energy levels, as well as the reduction in body and subcutaneous fat.

As used herein, the term “dietary acceptable” is intended to refer to, but is not limited to any food stuffs, vitamin, or mineral capable of being safely consumed, ingested, or eaten by an animal without harmful side effects.

As used herein, the term “supplement” is intended to refer to, but is not limited to a shake, a consumable oral strip, a consumable oral film, a beverage, a liquid, a gel, a tablet, a coated tablet, a capsule, a powder, a syrup, a suspension, a solution, a confectionery, a supplemental food, or any combination thereof.

As used herein, the term “dietary regimen” may include, but is not limited to, a combination of food and/or drink items that include the nutritional formulations or supplements described.

The term “mammal” includes, but is not limited to rodents, aquatic mammals, domestic animals such as dogs and cats, farm animals such as sheep, pigs, cows, and horses, and humans. Wherein the term mammal is used, it is contemplated that it also applies to other animals that are capable of the effect exhibited or intended to be exhibited, by the mammal.

As used herein, the term “blood sugar” is intended to refer to the amount or concentration of glucose present in a human or animal. It is also intended to refer to, but is not limited to the functional equivalent of the terms “blood sugar level”, “blood glucose level” or “blood glucose.”

As used throughout, ranges are used as shorthand for describing each and every value that is within the range. Any value within the range can be selected as the terminus of the range. When used, the phrase “at least one of” refers to the selection of any one member individually or any combination of the members. The conjunction “and” or “or” can be used in the list of members, but the “at least one of” phrase is the controlling language. For example, at least one of A, B, and C is shorthand for A alone, B alone, C alone, A and B, B and C, A and C, or A and B and C. As used herein, “and/or” means that the listed items are alternatives, but the alternatives also include any combination of the listed items.

All values contained throughout this application, including the claims are deemed to be approximate, whether or not the term “about” is used, unless specifically stated as exact.

### Description

Reduction of blood sugar has been shown to be effective in helping humans lose weight, reduce body fat, increase energy levels and prevent various diseases. Further, the health benefits associated with reduction of blood sugar is intended to coincide with healthy dietary habits and regular exercise.

It has been shown that GTF in yeast has similar effects to that of insulin in mammals. Over time, elevated blood sugar levels can lead to significant health problems associated with diabetes. GTF derived from yeast has been shown to reduce blood sugar levels in mammals, especially humans. While there are multiple types of yeast, Brewer's yeast has been found to be an effective source of GTF as it contains chromium. When combined with other vitamins, minerals and/or herbs GTF chromium has been shown to be particularly effective in reducing blood sugar levels, and is easily absorbed by the human body.

Glutamine plays a variety of roles in mammals including, but not limited to, amino acid and protein building as well as being a source of energy next to glucose. Specifically, L-Glutamine, any salt-thereof, or other compound thereof, is useful as a source of energy. L-Glutamine occurs naturally in foods such as beef, chicken, fish, eggs, dairy products, wheat, spinach, and beans. The L-Glutamine compound in the present invention may contain a wide variety of additional additives that are customarily incorporated by one skilled in the art of creating and synthesizing L-Glutamine. Such additives include, but are not limited to vitamins, sweetening agents, organic acids, coloring agents, flavoring agents, anti-wetting agents, fibers, electrolytes, minerals, nutrients, antioxidants, preservatives, aromas, humectants, natural plant extracts, and fruit extracts. In the present invention, L-Glutamine may be in free amino acid form or peptide form.

In preferred embodiments the nutritional formulation (composition) or dietary regime optimally contains dietary acceptable amounts of GTF chromium (e.g., from Brewer's Yeast); and L-Glutamine. In some presently preferred embodiments the nutritional formulation contains about 200 mcg of GTF chromium. In some presently preferred embodiments the nutritional formulation contains about 500 mg of L-Glutamine.

Additionally, the nutritional formulation (composition) or dietary regime can contain a dietary acceptable amount of pantothenic acid also known as pantothenate or Vitamin B₅. Pantothenic acid is commonly found most foods including meats, whole grains, vegetables such as broccoli and avocado, and cold-water fish ovaries. Other forms of pantothenic acid suitable for the present invention include calcium pantothenic acid which is stable in the digestive track, as it is a salt, and thus more easily absorbed by the body. Pantothenic acid by itself has a variety of known uses including but not limited to hair care, wound healing, weight control and blood sugar management.

The nutritional formulation (composition) or dietary regime may contain additional additives such as vitamins, sweetening agents, organic acids, coloring agents, flavoring agents, anti-wetting agents, fibers, electrolytes, minerals, nutrients, antioxidants, preservatives, aromas, humectants, natural plant extracts, and fruit extracts in order to increase absorption, prolong the shelf life of the supplement, improve taste and/or improve the supplement's appearance.

In some embodiments, the nutritional formulation or dietary regime also contains a dietary acceptable amount of Pantothenic Acid (Vitamin B₅), about 10 to 75 mg, preferably about 60 mg.

In some embodiments the nutritional formulation or dietary regime also contains a dietary acceptable amount of Thiamine (Vitamin B1), about 10 to 50 mg, preferably about 25 mg.

In some embodiments the nutritional formulation or dietary regime also contains a dietary acceptable amount of Riboflavin (Vitamin B2), about 10 to 75 mg, preferably about 10 to 50 mg.

In some embodiments the nutritional formulation or dietary regime also contains a dietary acceptable amount of Niacin (Vitamin B3) (e.g., as Niacinamide or nicotinic acid), about 10 to 75 mg, preferably about 25 mg.

In some embodiments the nutritional formulation or dietary regime also contains a dietary acceptable amount of Vitamin B₆, preferably about 25 to 75 mg.

Other optional ingredients suitable for inclusion in the nutritional formulation or dietary regime may include, but are not limited to, dietary acceptable amounts of compounds such as Folic Acid (about 200 to 400 mcg, preferably about 200 mcg); Cobalamin (Vitamin B₁₂) (about 15 to 50 mcg, preferably 25 to 50 mcg); Biotin (Vitamin H) (preferably about 100 to 200 mcg); Pantothenic Acid (Vitamin B₅) (about 25 to 75 mg, preferably about 60 mg); Paba (Para-aminobenzoic acid) (about 25 to 75 mg, preferably about 50 mg); Choline (about 25 to 75 mg, preferably about 50 mg); Inositol (about 25 to 75 mg, preferably about 50 mg); and cinnamon (preferably trace amounts).

### A Beverage

In some other preferred embodiments the nutritional formulation is in the form of a beverage or drink or a composition (e.g., powder(s)) that may be mixed with a liquid such as water or milk to form a beverage or drink. In these embodiments the nutritional formulation preferably contains about 10-50 mcg GTF Chromium. The nutritional formulation of these embodiments may also contain about 50-300 mg Glutamine.

The nutritional formulation for the beverage or drink/composition of these embodiments may also contain some or all of the following:

| Ingredient | Preferred amount | |
|---|---|---|
| Thiamine | 2-10 | mg |
| Riboflavin | 2-10 | mg |
| Niacinamide | 2-10 | mg |
| Pantothenic acid | 2-10 | mg |
| Vitamin B6 | 2-10 | mg |
| Folic acid | 25-100 | mcg |
| Choline | 2-10 | mg |
| Inositol | 2-10 | mg |
| Lecithin | ⅛ | teaspoon |
| bitter orange extract | dietary acceptable amount | |
| ground flaxseed powder | 100 | mg |
| banana fruit blend | 50-200 | mg |

Those of ordinary skill in the art will realize and appreciate, upon reading this description, that a particular beverage or drink or composition may include different and/or other ingredients. Those of ordinary skill in the art will also realize and appreciate, upon reading this description, that the ranges given are for an 8 oz. beverage or drink, but may also be used for a smaller or larger amount of liquid.

When in the form of a composition to be mixed into a drink or beverage, the composition is preferably in the form of a powder.

The following examples describe some of the preferred embodiments of the present technology without limiting the technology thereto.

### EXAMPLE 1

One embodiment of the present invention involves a nutritional formulation or dietary regime comprising the combination of:

about 200 mcg GTF chromium; and

about 500 mg of L-Glutamine.

### EXAMPLE 2

A second embodiment of the present invention involves a nutritional formulation or dietary regime comprising the combination of:

about 200 mcg GTF chromium;

about 500 mg of L-Glutamine; and

about 10 mg of Pantothenic Acid (Vitamin B₅).

### EXAMPLE 3

A third embodiment of the present invention involves a nutritional formulation or dietary regime comprising the combination of Example 1 with one or more of:

about 10 to 75 mg of Pantothenic Acid (Vitamin B₅); and

about 25 to 50 mg of Thiamine.

### EXAMPLE 4

A fourth embodiment of the present invention involves a nutritional formulation or dietary regime comprising any of the combinations of examples 1, 2 or 3 with about 10 to 50 mg of Riboflavin (Vitamin B₂).

### EXAMPLE 5

A fifth embodiment of the present invention involves a nutritional formulation or dietary regime comprising any of the combinations of examples 1, 2, 3 or 4 with about 10 to 75 mg of Niacin (as Niacinamide or nicotinic acid).

### EXAMPLE 6

A Sixth embodiment of the present invention involves a nutritional formulation or dietary regime comprising any of the combinations of examples 1, 2, 3, 4, or 5 with about 10 to 75 mg of Vitamin B₆.

### EXAMPLE 7

Other embodiments of the present invention involve nutritional formulations or dietary regimes of any of the combinations of the first six examples with one or more of the following:

about 50 to 200 mcg of Folic Acid;

about 10 to 50 mcg of Cobalamin (Vitamin B₁₂);

about 50 to 200 mcg of Biotin (Vitamin H);

about 10 to 75 mg of Pantothenic Acid (Vitamin B₅);

about 10 to 75 mg of Paba (Para-aminobenzoic acid);

about 10 to 75 mg of Choline;

about 10 to 75 mg of Inositol; and

trace amounts of cinnamon.

### EXAMPLE 8

One embodiment of the present invention involves a nutritional formulation or dietary regime in the form of a beverage or a composition such as a powder that may be mixed with a liquid such as water or milk to form a beverage, comprising the combination of: about 10-50 mcg GTF Chromium and about 50-300 mg Glutamine.

### EXAMPLE 9

A Ninth embodiment of the present invention involves a nutritional formulation or dietary regime comprising the combination of Example 8 with one or more of:

| Ingredient | Amount |
|---|---|
| Thiamine | about 2-10 mg |
| Riboflavin | about 2-10 mg |
| Niacinamide | about 2-10 mg |
| Pantothenic acid | about 2-10 mg |
| Vitamin B6 | about 2-10 mg |
| Folic acid | about 25-100 mcg |
| Choline | about 2-10 mg |
| Inositol | about 2-10 mg |
| Lecithin | about ⅛ teaspoon |
| bitter orange extract | dietary acceptable amount |
| ground flaxseed powder | about 100 mg |
| banana fruit blend | about 50-200 mg |

The foregoing is merely illustrative and not limiting, having been presented by way of example only. Although examples have been shown and described, it will be apparent to those having ordinary skill in the art that changes, modifications, and/or alterations may be made.

Although many of the examples presented herein involve specific combinations or elements, it should be understood that those acts and those elements may be combined in other ways to accomplish the same objectives.

As used herein, whether in the written description or the claims, the terms “comprising”, “including”, “having”, “containing”, “involving”, and the like are to be understood to be open-ended, that is, to mean including but not limited to. Only the transitional phrases “consisting of” and “consisting essentially of”, respectively, are closed or semi-closed transitional phrases with respect to claims.

1. A nutritional formulation for animal weight control, the nutritional formulation comprising:
about 200 mcg GTF chromium;
about 500 mg of L-Glutamine;
about 10 to 50 mg Thiamine;
about 10 to 75 mg Riboflavin;
about 10 to 75 mg Niacin;
about 10 to 75 mg Pantothenic acid;
about 10 to 75 mg Vitamin B6;
about 50 to 400 mcg Folic acid;
about 10 to 75 mg Choline, and about 25 to 75 mg Inositol;
about 10 to 50 mcg of Vitamin B12;
about 50 to 200 mcg of Biotin;
about 10 to 75 mg of Paba; and
a dietary acceptable amount of cinnamon.

2. The nutritional formulation according to claim 1 wherein the amount of Pantothenic acid is about 10 mg.

3. The nutritional formulation according to claim 1 wherein the amount of Thiamine is about 10 mg.

4. The nutritional formulation according to claim 1 wherein the amount of Riboflavin is about 10 to 50 mg.

5. The nutritional formulation according to claim 1, wherein the amount of Niacin is about 10 mg.

6. The nutritional formulation according to claim 1, wherein said Niacin is Niacinamide or nicotinic acid.

7. The nutritional formulation according to claim 1 further comprising about 50 to 200 mcg of Folic Acid.

8. The nutritional formulation according to claim 1, wherein the nutritional formulation is capable of being ingested by an animal as an oral supplement.

9. The nutritional formulation according to claim 8, wherein the animal is a mammal.

10. The nutritional formulation according to claim 1, wherein the formulation is in the form selected from the group consisting of: shakes, beverages, liquids, gels, tablets, coated tablets, capsules, powders, syrups, suspensions, solutions, confectioneries, oral strips, oral films, and food supplements.

11. The nutritional formulation of claim 1 further comprising one or more of:
about ⅛ teaspoon Lecithin,
a dietary acceptable amount of bitter orange extract,
about 100 mg ground flaxseed powder.

12. The nutritional formulation of claim 11 further comprising a fruit blend.

13. The nutritional formulation of claim 12 wherein the fruit blend comprises about 50-200 mg banana fruit blend.

14. A method for weight control in an animal, the method comprising:
orally administering a nutritional formulation according to claim 1.

15. A dietary regime comprising a nutritional formulation according to claim 1.
